(19) United States
(12) Patent Application Publication
(10) Pub. No.: US 2016/0367215 A1
(43) Pub. Date: Dec. 22, 2016

(54) METHOD FOR IN VIVO EVALUATION OF THE PHYSIOPATHOLOGICAL STATE OF A BIOLOGICAL TISSUE, AND ASSOCIATED DEVICE

(71) Applicants: Université François Rabelais, Tours (FR); Centre Hospitalier Régional Universitaire de Tours, Tours (FR); INSERM (Institut National de la Santé de la Recherche Médicale), Paris Cedex 13 (FR); Université Technologique de Compiègne, Compiègne (FR)

(72) Inventors: Joseph FOURNIER, Tours (FR); Marielle DEFONTAINE, Tours (FR); Frédéric PATAT, Saint-Cyr-sur-Loire (FR); Francis CANON, Lacroix-Saint-Ouen (FR)

(21) Appl. No.: 15/101,480

(22) PCT Filed: Dec. 4, 2014
(86) PCT No.: PCT/FR2014/053177

§ 371 Date: Jun. 3, 2016

(30) Foreign Application Priority Data
Dec. 4, 2013 (FR) 1362071

(51) Int. Cl. A61B 8/08; A61B 5/00

(52) U.S. Cl. CPC A61B 8/08; A61B 5/0053; A61B 5/4523; A61B 5/4533; A61B 5/4519; A61B 5/458; A61B 5/4585; A61B 5/4595

(57) ABSTRACT

Disclosed is a method for evaluating the physiopathological state of a biological tissue situated near a joint, the method including: measurement of the force and/or of the movement to which the joint is subjected; emission of an ultrasound wave into the tissue; simultaneous acquisition of the ultrasound data, which are received after propagation of an ultrasound wave emitted into the tissue, and of the force and/or movement data of the joint; and processing of the data. Also disclosed is a device for implementing the method.

### FIELD OF THE INVENTION

The present invention relates to a method for the in vivo evaluation of the physiopathological state of a biological tissue situated close to a joint. More precisely, the present invention makes it possible to evaluate the mechanical properties of said biological tissue and variations thereof. The present invention also relates to a non-invasive medical device for the in vivo evaluation of the physiopathological state of a biological tissue situated close to a joint.

### PRIOR ART

Musculoskeletal disorders are among the main causes of pain and handicap in adults. Generally related to hyper-use, these disorders are often associated with sporting activities or work activities. They are mainly ascribed to traumatic overload at one point, such as in the case of sprint races, or to repeated microtrauma such as in the case of long-distance races.

Tendon pathologies represent a large proportion of musculoskeletal disorders. They may affect 30% to 50% of the physically active population. Among them, Achilles tendinopathy is particularly frequent in the male population aged from 30 to 50 years. Relating to the general population, the incidence of Achilles tendinopathy, also referred to as calcaneal tendinopathy, is measured at 2 per 1000, and 35% of cases would be related to sporting practice. Incidence among high-level athletes is even much higher.

Tendinopathy, which may be asymptomatic for a long time, greatly and at length affects the quality of life and the productivity of persons. Achilles tendon ruptures generally occur on pathological tendons and tendinopathies are thus often diagnosed after the event. Furthermore, healing of tendon lesions is a complex and poorly-known phenomenon that is accompanied by changes in mechanical properties: the scar tissue is thus often mechanically inferior to the original tissue.

It therefore appears essential, from a societal point of view to limit so-called professional diseases, from a medical point of view to evaluate and monitor the functional capacities of tendons, and from a sporting point of view to prevent tendinopathies and reductions in sporting performance, to be able to prevent and observe the development of tendon lesions. Medical care for tendinopathies, which are often asymptomatic, currently takes place late and clinical diagnosis is then supplemented by means of medical imaging examinations. Current techniques for evaluating tendon pathologies are based mainly on echography and MRI (magnetic resonance imaging). These imaging methods provide important morphological information on the state of the tendon in a static condition but do not, in standard practice, give information on the state of the tendon and its variations over time (i.e. in dynamic condition). However, the tendon is a dynamic structure par excellence since it allows movement of the human body by connecting a muscular tissue to a bone structure. A tendon must therefore be stressed statically, by isometric contraction of the muscle for example, or dynamically, for example by eccentric or concentric contraction of the muscle. Profound knowledge of tendon pathologies therefore requires knowing the dynamic properties and not only the static properties of the tendon. Furthermore, some obscure tendinopathies are not detectable through conventional imaging techniques. The aforementioned methods are also limited by the time necessary for acquiring images.

There does not exist at the present time any simple method, usable in clinical practice, for evaluating the physiopathological state of a tendon by means of these mechanical properties, which are known to be directly related to the state of health and to the elongation capacities of the tendon. A simple evaluation accessible to the clinician would enable the latter better to direct his therapeutic decisions and to advise his patients for dealing with these pathologies.

There therefore exists a real need in orthopaedic surgery, in sports medicine and in occupational medicine, for quantification and measurement of mechanical and in particular viscoelastic properties of the Achilles tendon in order to prevent tendon lesions in a healthy subject and where necessary to monitor changes in a pathological subject. Medical imaging techniques provide interesting qualitative information on the state of the tendon but no current method or device affords quantitative monitoring of the physiopathological state of the tendon combining measurement of forces suffered by the tendon and knowledge of its mechanical properties.

A person skilled in the art, knows, by means of the patent FR 01 13327, a method for determining the state of a material at a given instant, comprising a step during which the value is calculated of at least one parameter extracted from the ultrasound signal received after propagation of an ultrasound wave emitted by said material. This method describes a technique for knowing the state of tension of a material at a given instant and during a given exercise from ultrasound measurements. However, this method does not make it possible to combine, with these ultrasound measurements, measurement of the forces imposed on the tendon or suffered by the tendon, in particular by means of an ergometer. Likewise, this method of the prior art does not ensure repeatability of the method over time. Furthermore, this method does not have any clinical interest since knowledge of the state of tension of a biological tissue does not presage the rupture or the physiopathological state of said tissue.

Thus, by way of illustration, if the concern is with the state of a cord, the invention as described in the patent FR 01 13327 will make it possible to know its state of tension at a given moment by means of the measurement of ultrasound parameters. This method provides an instantaneous photograph of the state of tension but this will not make it possible to predict the stresses as from which the cord may break. For this purpose a destructive test will be necessary, and it will be necessary to measure the state of tension at the moment of rupture. Such a test is obviously inapplicable to the medical field.

Thus at the present time a normal application method allowing the simultaneous measurement of the mechanical properties of a tendon and the force imposed on said tendon or suffered by said tendon is not available.

One of the objectives of the present invention is therefore to enable the clinician to obtain information on the physiopathological state of an Achilles tendon, by associating, with the measurements made on the biological tissue by means of ultrasound waves, the measurements of the actions (force, stresses, movement, deformation, etc.) imposed on the joint or suffered by the joint. Thus, in order to resume the example of the cord, the present invention makes it possible, by associating, over time, the ultrasound measurements made on the cord with the measurements of the stresses, to be able to predict the state of the cord outside the measurement range.

The present invention proposes a non-destructive dynamic evaluation. By comparing the variation in the stresses imposed on the joint or suffered by the joint with the variation in the ultrasound parameters collected, the present invention makes it possible to have knowledge about the variation in the mechanical properties according to the stresses. The invention thus makes it possible to predict the change in the mechanical properties of the tissue according to the stresses. Measuring the two parameters (stresses and ultrasound parameters collected) makes it possible to dispense with knowledge of the tension state.

Another objective of the invention is based on the simplicity of use, the ease of transport and the energy autonomy of the device used in the method of the present invention. This is because there does not exist at the present time any portable device, usable daily in clinical practice, making it possible to know the physiopathological state of a biological tissue. The invention proposes to overcome this lack by proposing a portable and easily transportation integrated device.

The present invention thus makes it possible to characterise in vivo a biological tissue from a not morphological but biomechanical point of view, non-invasively by means of a portable device allowing simultaneously the measurement of the parameters of propagation of an ultrasound wave through a tissue and measurement of the force and/or joint movement. The present invention is therefore particularly useful both for aiding diagnosis and dealing with tendon pathologies but also for screening for and diagnosing said pathologies and monitoring the change in the physiopathological state by virtue of knowledge of the mechanical properties of the tendon.

### SUMMARY

The invention therefore relates to a method for the non-invasive evaluation of the physiopathological state of a biological tissue situated close to a joint, comprising the emission of an ultrasound wave in said tissue, from an ultrasound source, the simultaneous acquisition of at least one parameter extracted from the ultrasound signal received after propagation of the ultrasound wave, and at least one force and/or joint-movement data item, and processing of the information simultaneously acquired; said method not being an ultrasound imaging method.

According to one embodiment, said evaluation method further comprises a first step of applying a force and/or a predefined joint movement.

According to one embodiment, said joint is a joint of the human body comprising a main degree of freedom, preferably said joint is an elbow joint, a knee joint, an ankle joint, a metacarpophalangeal joint, a metatarsophalangeal joint or an interphalangeal joint.

According to one embodiment, the biological tissue studied is a tendon, a muscle, a ligament, a nerve or the skin, preferentially an Achilles tendon, a quadricipital tendon or the brachial triceps tendon.

According to one embodiment, the processing of said data simultaneously acquired comprises the extraction of at least one ultrasound parameter chosen from the speed of propagation of the ultrasound wave, the attenuation of the ultrasound wave at the point of reception of the wave, the amplitude of the ultrasound wave at the point of reception of the wave, the frequency of the ultrasound wave at the point of reception of the wave, or the change in the frequency spectrum of the ultrasound wave; and the correlation between said ultrasound parameter and the force and/or joint-movement data measured.

The invention also relates to a device for evaluating the physiopathological state of a biological tissue situated close to a joint, for implementing the method according to the present invention.

According to one embodiment, said evaluation device comprises:
a means for measuring the force and/or the joint movement such as, non-limitatively, an ergometer, a force sensor, a movement sensor, an accelerometer or a dynamometer;
an ultrasound sensor comprising at least one sending transducer and at least one receiving transducer;
a system for the simultaneous acquisition of the ultrasound data received after propagation of an ultrasound wave emitted in said tissue and the force and/or joint-movement data; and
a system for processing said data.

According to one embodiment, said device further comprises a means for applying a force and/or a predefined joint movement, such as for example an actuator, a hydraulic actuator or a motor.

According to one embodiment, said device comprises, so as to be portable, integrated and non-invasive, at least one means for measuring force and/or a joint movement, at least one ultrasound sensor, at least one system for the simultaneous acquisition of ultrasound data and force and/or joint-movement data, at least one system for processing said data and at least one self-contained energy source; an ultrasound sensor being placed on the skin opposite the biological tissue and a means for measuring a force and/or a joint movement being situated at the joint being acted on.

According to one embodiment, a sending transducer and a receiving transducer are aligned along the functioning axis of the tissue being studied.

According to one embodiment, the acquisition and processing system comprises an acquisition card, a microprocessor, a data-storage space and at least one item of data-processing software.

According to one embodiment, the device further comprises a module for displaying data after processing and/or a data-transmission means.

Definitions

In the present invention, the following terms are defined as follows:

“joint with a main degree of freedom” relates to any joint of the human body the predominant movement of which comprises a degree of freedom (e.g. the elbow, ankle or knee joint, or the metacarpophalangeal, metatarsophalangeal or interphalangeal joints).

“ankle joint” relates to the ankle joint or talocrural joint that stresses, among other things, when functioning, the Achilles tendon.

“knee joint” includes the patellofemoral joint and the internal and external femorotibial joint, and stresses in particular, during functioning, the patellar ligament and the quadricipital tendon.

“correlation”, within the meaning of the present invention, relates to the study of the relationship between two parameters; by means of a statistical study well known to persons skilled in the art and/or a graphical representation of one of the parameters as a function of the other, and/or by means of any other method that a person skilled in the art would judge opportune.

“concentric contraction” relates to a contraction causing a controlled shortening of the muscle.

“eccentric contraction” relates to a contraction causing a controlled elongation of the muscle.

“isometric contraction” relates to a contraction characterised by an absence of movement. It is the contraction of the muscle for resisting a force without movement of the joint.

“ergometer” designates a physical-exercise machine that consists of making the user reproduce a defined exercise; the ergometer according to the present invention comprises a means for measuring a force and/or a movement of the joint.

“joint force and/or movement” designates the force and/or movement imposed on the joint by any external means within the capability of a person skilled in the art or the force and/or movement suffered by the joint through muscle action.

“ligament” relates to an anatomical formation joining two bone structures.

“means for measuring a force and/or a movement” designates any means such as a sensor, a gauge, an accelerometer, a dynamometer or an ergometer for measuring joint force and/or movement.

“physiopathology or physiopathological state” relates to the study of disturbances to the normal operating mode of parts of the human body. Thus a physiopathological state corresponds to the state of physiological disturbance of the biological tissue. Knowing or evaluating the physiopathological state thus consists of knowing and studying the changes to the functions of the organism during an illness, such as for example a tendinopathy.

“tendon” relates to an anatomical formation producing the junction between a muscle and a bone structure.

“biological tissue situated close to a joint” relates to a tissue such as for example a muscle, a ligament, a tendon, a nerve or the skin” exercised during the movement of the bone parts of the joint.

### DETAILED DESCRIPTION

The present invention relates to a method for the in vivo evaluation of the physiopathological state of a biological tissue. Said method comprises, non-invasively, the simultaneous measurement of the joint force and/or movement and at least one parameter calculated from an ultrasound signal received after propagation of an ultrasound wave in said biological tissue.

The invention therefore relates to a method for the non-invasive evaluation of the physiopathological state of a biological tissue situated close to a joint, comprising the emission of an ultrasound wave in said tissue, from an ultrasound source, and the simultaneous acquisition of at least one parameter extracted from the ultrasound signal received after propagation of the ultrasound wave, and from at least one joint force and/or movement data item, and the processing of the information simultaneously acquired.

Said method according to the present invention not being an ultrasound imaging method.

The present invention relates to a method for evaluating the physiopathological state of a biological tissue by means of the measurement of the mechanical properties of said biological tissue under stress.

In one embodiment, said biological tissue is a biological tissue of a mammal, preferably a human being.

In one embodiment, said biological tissue is any biological tissue, preferentially a muscle tissue, an epithelial tissue, a nerve tissue or a conjunctive tissue, even more preferentially a tendon, a muscle, a ligament, a nerve or the skin.

In one embodiment, said biological tissue is situated at a joint and/or close to the skin in order to facilitate the taking of measurements by ultrasound wave and the recovery of information corresponding specifically to the tissue being studied.

In one embodiment, said joint is any joint of a mammal or of a human being, preferably a joint with one degree of freedom, even more preferentially an ankle, knee or elbow joint, a metacarpophalangeal joint, a metatarsophalangeal joint or an interphalangeal joint.

In a preferential embodiment, the present invention relates to the evaluation of the physiopathological state of a tendon, muscle or ligament at an elbow joint, a knee joint, an ankle joint, a metacarpophalangeal articulation, a metatarsophalangeal articulation or an interphalangeal articulation.

In an even more preferential embodiment, the present invention concerns the evaluation of the physiopathological state of an Achilles tendon, a patellar ligament, a quadricipital tendon or a brachial triceps tendon.

The present invention comprises the measurement of the force and/or movement to which the joint is subjected. The force and/or movement may be imposed by means of an external device or means or by the subject himself, for example during a movement.

It has been shown by the applicant, without wishing to be bound by a theory, that an excellent correlation is observed between certain parameters calculated from the ultrasound signal received after propagation of an ultrasound wave in a biological tissue, such as for example the speed of propagation of the wave, and the stresses applied to said tissue.

It is therefore important for the method of the invention to comprise a step of measuring the joint force and/or movement, or in an equivalent manner for a person skilled in the art, the stress and/or deformation or the force and/or position.

In one embodiment, the present invention comprises a first step of applying a predefined force and/or movement.

In one embodiment, the means for measuring the force and movement may comprise two distinct measurement means, one for measuring the force and one for measuring the movement, or a combined measurement means.

In one embodiment, the means for measuring the joint force and/or movement may be any means known to persons skilled in the art such as an accelerometer, a dynamometer, a force sensor or a movement sensor.

In one embodiment, the invention comprises a means for applying a predefined joint force and/or movement, which may be an actuator (in particular a hydraulic actuator) or a motor, well known to persons skilled in the art for causing the mobility of a limb or a joint. In one embodiment, said means is capable of making the user reproduce a natural movement and/or measuring a predefined force and/or movement. Said means of the present invention is dedicated to the biomechanical characterisation of a joint and makes it possible to stress the tissue by imposing on said joint a force and/or a movement. In one embodiment, said means comprises a rotation axis coinciding with the rotation axis of the joint being stressed.

In one embodiment, the invention comprises a means for measuring the joint force and/or movement and the means for applying a predefined joint force and/or movement.

In one embodiment, the subject, and in particular his joint of interest, is free and makes a joint movement, giving rise to a joint force and/or movement. Said joint force and/or movement is then measured by means of a measuring means according to the invention. In one embodiment, the invention comprises a means for measuring the joint force and/or movement without the application of a force and/or movement. Said movement and/or said force being imposed by the subject.

In one embodiment, the means for measuring the joint force and/or movement is fixed to the joint being studied by any fixing means within the capability of a person skilled in the art, such as straps for example.

In one embodiment, the ergometer is fixed to the joint being studied by any fixing means within the capability of a person skilled in the art, such as straps for example.

In one embodiment, said fixing means is adjustable in order to accommodate subjects with varied anthropometric characteristics.

In one embodiment, the ergometer comprises a rotation axis coinciding with the rotation axis of the joint being stressed. The ergometer makes it possible to measure a joint force and/or movement.

As is known to persons skilled in the art, the force may be converted into a moment of force, knowing the distance between the rotation axis of the joint and the force sensor of the ergometer.

In an embodiment where the Achilles tendon is studied, the subjects are installed on an ergometer comprising a platform guided in rotation on an axis corresponding to the axis of the ankle joint. The joint angles of the knee and ankle are adjusted to predefined values, preferentially respectively 120° and 90°. In this embodiment, the foot of the subject is secured to the platform by means of a shoe and straps, taking care to make the rotation axis of the ankle coincide with the rotation axis of the drive.

In one embodiment, the ergometer of the present invention makes it possible to perform tests in dynamic condition (concentric contraction of the muscle or eccentric contraction of the muscle) or in static condition (isometric contraction of the muscle). It should be noted that, in the embodiment of the invention where an eccentric muscle contraction is performed, the invention comprises a means for exerting on the joint an external force with a direction opposite to and greater than the muscle force produced.

In one embodiment, the ergometer comprises a force sensor and/or position sensor (making it possible to know the movement) securely fixed to the ergometer.

The data issuing from the force sensor and/or from the position sensor are acquired by means of an acquisition system allowing the simultaneous acquisition of said data and the data issuing from the ultrasound sensor.

The present invention comprises the emission of an ultrasound wave in a tissue. This is because the present invention allows the in vivo characterisation of a biological tissue from a biomechanical point of view by measuring the mechanical properties of a biological tissue when it is stressed. This measurement of the mechanical properties of a biological tissue is in particular made by propagating an ultrasound wave in said tissue. This measurement of the mechanical properties of a biological tissue is not obtained by ultrasound imaging.

To make these measurements an ultrasound sensor is placed on the skin facing the tissue being studied so that the distance between sensor and tissue being studied is as small as possible or so that the propagation of the ultrasound wave is the best possible (by acting for example on the angle). It has been shown that the measurements made are not affected by the passage of the wave through the skin.

In one embodiment, the distance between sensor and tissue being studied is between 1 millimetre and 25 centimetres, preferably between 5 millimetres and 5 centimetres.

In one embodiment, the face of the sensor in contact with the skin is manufactured from a biocompatible material, preferably a biocompatible silicone.

In one embodiment, the sensor is adapted to the tissue to be studied so that the form of the face of the sensor in contact with the skin corresponds to the morphology of this body part, and thus generally the sensor is slightly concave.

In one embodiment, the ultrasound sensor comprises at least one emitting transducer, preferably 1 to 50 emitting transducers, more preferentially 1, 5, 10, 15, 20, 25, 30, 35, 40, 45, 50 emitting transducers and at least one receiving transducer from preferably 1 to 100 receiving transducers, more preferably 1, 5, 10, 15, 20, 25, 30, 35, 40, 45, 50, 60, 70, 80, 100 receiving transducers. The signals coming from this emitter or emitters that have travelled through said tissue are thus received by means of said receiver or receivers. Preferably, the sending and receiving transducers are conventional transducers well known to persons skilled in the art.

In one embodiment, the at least one sending transducer and the at least one receiving transducer are aligned along the main axis of the tissue being studied, i.e. the axis of the fibres of the tissue or the functioning axis of the tissue.

In one embodiment, the at least one sending transducer and the at least one receiving transducer are included in the ultrasound sensor. In one embodiment, the distance between the sending transducer and at least one receiving transducer is fixed and predetermined

In one embodiment, the ultrasound sensor emits short ultrasound pulses that enter the tendon at the critical angle in accordance with the Snell-Descartes law, and the ultrasound waves then propagate along the fibres and are added in phase at the receiving transducers of the sensor.

The presence of a plurality of receivers aligned with the at least one sender ensures better reliability of the measurements since the travel time of the wave (and therefore the speed of propagation of the waves since the distance is fixed and determined between the sender and its receiver) between the sender and its receiver is calculated independently in order to obtain a reliable mean propagation speed.

In one embodiment, the emitter/receiver distances are chosen so as to receive first the head wave, which is the longitudinal wave propagating at the skin/tissue interface.

In one embodiment, the measurement of the speed of propagation of the head wave is based on the digital detection of the first maximum associated with a technique of interpolation between receivers that is well known to persons skilled in the art.

In one embodiment, the sensor is fixed opposite the tissue being studied. This holding of the sensor without intervention by the operator guarantees independence of the measurement with respect to the operator.

In one embodiment, the angle formed between the ultrasound emitter and at least one ultrasound receiver is between 60° and 120°, preferentially between 70° and 110°, preferentially 80°; the sender being positioned symmetrically with the receiver with respect to a plane perpendicular to the axis of the fibres.

In a preferential embodiment, the frequency of repetition of the emission of waves by the source (PRD: “Pulse Repetition Frequency”) is between 0.1 kHz and 100 kHz, preferably between 1 kHz and 10 kHz.

In a preferential embodiment, the frequency of the ultrasound waves emitted by the sensor is between 0.35 and 20 megahertz, preferably between 0.6 and 1.6 megahertz, even more preferentially between 1 and 1.4 megahertz.

Without wishing to be bound by any theory, it has been shown by the applicant that the speed of propagation of the wave has excellent correlation with the actions (force, stress, movement, deformation) applied to the tissue. The perspective is then opened up of observing the variations in the mechanical properties of the tissues at a very short timescale, around 1 millisecond.

The present invention comprises the simultaneous acquisition of the ultrasound data received after propagation of an ultrasound wave emitted in said tissue and force and/or movement data coming from the means for measuring the force and/or movement of the joint.

In one embodiment, the recording of the data from the means for measuring the joint force and/or movement and the data extracted from the ultrasound sensor are perfectly simultaneous in order to be able, after processing, to obtain quantitative results coupling these data.

In one embodiment, the data acquisition means comprise the electronic cards providing the functioning of the sensors of the means for measuring the joint force and/or movement and transducers (emitters and receivers) of the sensor.

In one embodiment, the sensor and the means for measuring the joint force and/or movement are connected to the acquisition system by means of a wireless connection or a wired connection, preferably by means of a wired connection.

The present invention comprises the processing of the ultrasound data received after propagation of an ultrasound wave emitted in said tissue and force and/or movement data coming from the means for measuring the joint force and/or movement.

The information on this wave after passing through said tissue is recovered by the extraction of at least one parameter chosen from:
i. the speed of propagation of the ultrasound wave in said tissue (or equivalently for a person skilled in the art the travel time of the ultrasound wave over a predetermined distance in said tissue);
ii. the amplitude of the ultrasound wave at the point of reception of the ultrasound signal;
iii. the attenuation of the ultrasound wave at the point of reception of the ultrasound signal;
iv. the mean frequency of the ultrasound wave at the point of reception of the ultrasound signal;
v. the maximum frequency of the ultrasound wave at the point of reception of the ultrasound signal; and/or
vi. the variation in the frequency spectrum of the ultrasound wave.

In a preferential embodiment, the speed of propagation of the ultrasound signal is recovered.

In one embodiment, the parameter is not the amplitude of the ultrasound wave at the point of reception of the ultrasound signal.

In one embodiment, the processing of said data comprises the extraction of at least one ultrasound parameter chosen from the speed of propagation of the ultrasound wave, the attenuation of the ultrasound wave at the point of reception of the wave, the amplitude of the ultrasound wave at the point of reception of the wave, the frequency of the ultrasound wave at the point of reception of the wave, or the modification to the frequency spectrum of the ultrasound wave; then the correlation between the chosen parameter and the force and/or the joint movement.

The present invention does not comprise the extraction of at least one ultrasound parameter for purposes of medical imaging.

In one embodiment, the acquisition and processing system comprises an acquisition card, a microprocessor, a data-storage space, and at least one item of data-processing software. In one embodiment, the acquisition and processing system further comprises a module for displaying the data after processing and/or a means for transmitting the data.

In one embodiment, the processing of the data generated by the sensor and the means for measuring the joint force and/or movement is done by means of software stored by the data-storage space.

In one embodiment, the software enables:
i. the sending of the ultrasound;
ii. the establishment of the force or movement required;
iii. the processing of the signals coming from the receiving transducers;
iv. the processing of the signals coming from the sensors of the ergometer;
v. the display on the display module of the data obtained.

Said software was registered with the APP under the application number IDDN.FR.001.470027.000.S.P.2013.000.21000.

In one embodiment, the data processing system also comprises a data display module.

In one embodiment, the speed of ultrasound propagation as a function of the force, the deformation or the torque developed at the joint is preferentially displayed on the display module.

The present invention also relates to a device for evaluating the physiopathological state of a biological tissue able to implement the method of the present invention.

More specifically, the present invention also relates to a device for evaluating the physiopathological state of a biological tissue situated close to a joint, comprising:
a means for measuring the joint force and/or movement;
an ultrasound sensor comprising at least one sending transducer and at least one receiving transducer;
a system for the simultaneous acquisition of the ultrasound data received after propagation of an ultrasound wave emitted in said tissue and the joint force and/or movement data; and
a system for processing said data.

In one embodiment, the evaluation device comprises, in a portable, integrated and non-invasive manner, the means for measuring a joint force and/or movement, the ultrasound sensor, the acquisition system, the processing system and a self-contained energy source; the ultrasound sensor being placed on the skin facing the biological tissue and the means for measuring a joint force and/or movement being situated at the joint being acted on.

In one embodiment, the at least one sending transducer and the at least one receiving transducer of the sensor are aligned along the functioning axis of the tissue being studied.

In one embodiment, the acquisition and processing system comprises an acquisition card, a microprocessor, a data storage space, and at least one item of data-processing software. In one embodiment, the acquisition and processing system further comprises a module for displaying the data after processing and/or a data-transmission means.

In one embodiment, the device of the present invention comprises a battery providing a sufficient self-contained energy supply for the device for making a plurality of measurements.

In one embodiment, the device of the present invention comprises a remote control for directing the device at a distance.

In one embodiment, the device of the present invention comprises means (USB, WiFi, Bluetooth, etc.) for transferring and storing the data on external media (video monitors, computers, etc.).

In one embodiment, the means for measuring a joint force and/or movement is positioned at the joint being studied and comprises a force sensor and/or a position sensor (making it possible to know the movement). An ultrasound sensor is fixed opposite the tissue being studied. Said sensor and said means for measuring a joint force and/or movement are connected to the acquisition and processing system and to a battery integrated in the device. The assembly being secured and easily transportable.

In one embodiment, the ergometer is positioned at the joint being studied and comprises a force sensor and/or a position sensor (making it possible to know the movement). An ultrasound sensor is fixed opposite the tissue being studied. Said sensor and said ergometer are connected to the acquisition and processing system and to a battery integrated in the device. The assembly being secured and easily transportable.

The medical method and device of the present invention can be used in the context of numerous applications.

The device according to the present invention is intended to measure not the tension but the state of health or physiopathological state of the tension according to its mechanical properties. It integrates in the same apparatus, simple to use, of the various components necessary for determining the mechanical properties and makes use thereof possible in clinical practice.

In one embodiment, the method of the present invention is used for diagnosis purposes in the service of a clinician in order to monitor the change in a pathology (tendinopathy, collagen pathology, etc.).

In one embodiment, the device of the present invention is used as a diagnosis tool in the service of a clinician in order to monitor the change in a pathology (tendinopathy, collagen pathology, etc.).

In one embodiment, the method of the present invention can serve as a method for evaluating the physiopathological state of a tissue in kinesiotherapy.

In one embodiment, the device of the present invention can serve as a kinesiotherapy evaluation tool.

In one embodiment, the method of the present invention is used for prevention purposes for the use of private individuals in order to evaluate changes in the mechanical properties of biological tissues and to predict risks of lesions.

In one embodiment, the device of the present invention is used as a prevention tool for the use of private individuals in order to evaluate changes in mechanical properties of biological tissues and to predict risk of lesions.

In one embodiment, the method of the present invention is used for monitoring purposes in order to monitor change in the mechanical properties of biological tissues of professional or amateur sportspersons.

In one embodiment, the device of the present invention is used as a monitoring tool in order to monitor change in the mechanical properties of biological tissues of professional or amateur sportspersons.

In one embodiment, the method of the present invention can serve for evaluating devices suited to sportspersons, such as for example novel shoes or novel floor coverings.

In one embodiment, the device of the present invention can serve as a tool for evaluating devices suited to sportspersons, such as for example novel shoes or novel floor coverings.

In one embodiment, the device according to the invention is not an ultrasound elastograph and does not use an ultrasound elastography method.

### BRIEF DESCRIPTION OF THE FIGURES

FIG. 1 is a schematic representation of the medical device of the present invention in an embodiment where the device is applied to the study of the physiopathological state of the Achilles tendon.

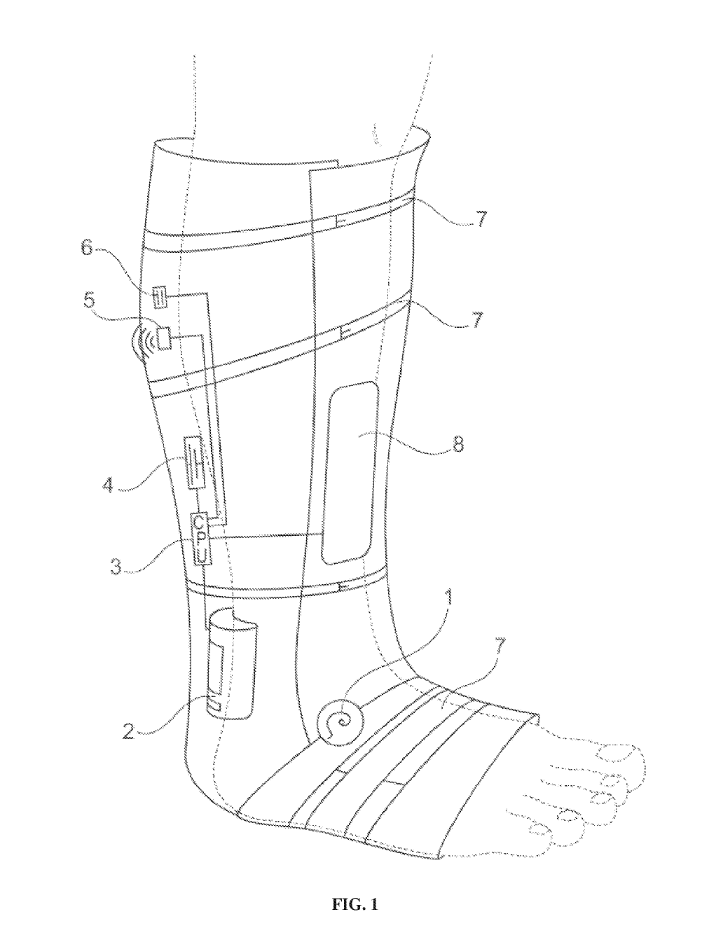

### REFERENCES

1—Ergometer-accelerometer,
2—Ultrasound transducer,
3—Microprocessor responsible for controlling the sensor and the processing of the signal,
4—Self-contained power supply,
5—WiFi connection,
6—USB connection,
7—System for attaching and adjusting the device,
8—Screen displaying the results.

### EXAMPLES

The present invention will be understood better from a reading of the following examples, which illustrate the invention non-limitatively.

### Example 1

A medical device evaluating the mechanical properties of the Achilles tendon and variations therein in the course of the contraction of the triceps surae has been developed. This apparatus has been used for in vivo clinical investigations under dynamic conditions. The preliminary results obtained in these studies showed excellent sensitivity of the ultrasound measurement to the change in stress in the tendon during an effort.

Equipment

Said device comprises, in integrated manner
(i) an ultrasound transducer 2 intended to measure the speed of propagation of the ultrasound wave in axial transmission, said transducer being fixed parallel to the tendon fibres;
(ii) an ergometer 1 for the combined acquisition of force and position data on the joint of the lower limb on which the tendon being studied is the effector;
(iii) a microprocessor 3 for processing the signals generated by the transducer and the ergometer; and extracting the data corresponding to the viscoelastic properties of the tendon being studied;
(iv) a module 8 for displaying and storing these data and the connections 5, 6 for transferring to other media such as a graphics tablet or any other video monitor;
(v) a self-contained energy source 4 of the battery type; and
(vi) straps 7 for fixing the various components of the device.

More precisely, said device comprises:
1. An ergonomic ultrasound sensor known to persons skilled in the art (such as those sold by the company Vermon). The front face, slightly concave (R=30 mm) for matching the curvature of the tendon, is manufactured from biocompatible silicone;
2. An electronic module for the sensor comprising:
   a. a card emitting wide-band pulses (>2 MHz)—180 Vpp,
   b. 5 reception cards (4 digitisation channels/card)—20 parallel digitisation channels,
   c. 1 microcontroller card (386 CPU),
   d. 1 USB2 control module,
   e. 1 Ethernet control module.
3. At least one item of dedicated software performing the following functions:
   a. configuration of the electronic module for measurement and transfer of the configuration to the electronic module (Ethernet link),
   b. recovery of the raw ultrasound data (USB2 connection) and recording in Excel files,
   c. opening of the Excel files and representation of the ultrasound signals/auxiliary inputs,
   d. detection of the first echo (head wave) on the ultrasound signals,
   e. calculation of the sender/receiver propagation speeds,
   f. recording of the processed data;
4. An RF remote control for sending the various operating modes of the module;
5. An Ethernet cable;
6. A 0V/+6V battery and 1 pair of suitable cables;
7. Two 0V/−10V and 0V/+10V batteries;
8. An isolating transformer to medical standards, 1000V;
9. A display module;
10. A data storage module;
11. An ergometer comprising a pedal drive with a force sensor and a position sensor (making it possible to know the movement);
12. An electrical module for the ergometer comprising:
   a. a 60 watt, 24V/2.5 A TXL 160-24S (Traco Power) power supply,
   b. a Data Translation DT 9800 acquisition card,
   c. an electronic card for conditioning the force sensor,
   d. an electronic card for conditioning the position sensor.

Methods

The study was carried out using a male population of 40 healthy volunteers aged from 18 to 60 years. The ultrasound speed measurements were carried out during two sessions spaced apart by four weeks in order to measure the medium-term reproducibility. At each session, two tests were carried out in order to evaluate the short-term reproducibility. Each test in each session consisted of three sets of measurements in order to evaluate the precision of the measurements.

The volunteer is installed and kept in position seated with his knee bent at 120°. During the warming-up phase prior to the calibrated exercise, the subject is familiarised with the production of the muscular contraction in isometric plantar flexion. He is requested to make the contractions greater and greater before measurement of the maximum voluntary contraction (MVC). The warming-up phase is necessary for any exercise related to the measurement of the viscoelastic properties of the tendons. This warming-up phase comprises first of all with one minute of angular movements of the ankles concerned, followed, after the installation of the device of the present invention including the ergometer and ultrasound transducer, by the performance of 10 sub-maximum isometric contractions and 3 maximum contraction (MVC) tests of the plantar flexors. The MVC is measured when the subject is deemed to have clearly understood the exercise.

Three tests will be carried out, the average of which is calculated. The exercise required is broken down into 5 phases over approximately 15 seconds of recording: this exercise will be explained to the patient and carried out without ultrasound measurement initially in order to ensure a good understanding of the exercise; once the exercise has been learned, it will be repeated conjointly with the ultrasound measurement.
1. Maintenance 20% MVC (1-2 seconds): the subject will have to make an isometric contraction in order to maintain a force corresponding to 20% of his MVC for two seconds (triggering of the acquisition);
2. Ramp 20%-80% MVC (3-5 seconds): the subject will have to make, in three to five seconds, a plantar flexion in order to achieve 80% MVC;
3. Maintenance 80% MVC (2 seconds): the subject will be requested to maintain this force for 2 seconds;
4. Ramp 80%-20% MVC (3-5 seconds): the subject will have to relax the plantar flexor contraction force in 3-5 seconds in order to return to a force of 20% of the MVC;
5. Maintenance 20% MVC (1-2 seconds): then maintenance of this force for two seconds (stoppage of the acquisition).

During the exercise described above, the ultrasound signals and the joint force and movement signals will be recorded simultaneously. The instructions to perform the exercise will be given orally, with compliance with the various times mentioned above and encouragement of the subject.

Results

From the ultrasound speeds measured, the intra-class correlation coefficients were calculated and the following results obtained.

| | | Initial 20% MVC | | | 80% MVC | | |
|---|---|---|---|---|---|---|---|
| 1^{st} Session | test 1 | 0.993 | 0.846 | 0.855 | 0.995 | 0.772 | 0.791 |
| | test 2 | 0.989 | | | 0.975 | | |
| 2^{nd} Session | test 1 | 0.993 | 0.915 | | 0.997 | 0.873 | |
| | test 2 | 0.993 | | | 0.992 | | |

| | | Final 20% MVC | | |
|---|---|---|---|---|
| 1^{st} Session | test 1 | 0.969 | 0.860 | 0.873 |
| | test 2 | 0.984 | | |
| 2^{nd} Session | test 1 | 0.975 | 0.927 | |
| | test 2 | 0.995 | | |

The coefficients of correlation between sessions are greater than 0.79, the coefficients of correlation between tests are greater than 0.77 and the coefficients of correlation between takings of measurements are greater than 0.97.

These results show that the intrapersonal variability is very much less than the interpersonal variability. There therefore exists an acoustic signature particular to each tendon. This makes it possible to conclude that it is possible to isolate a subject in a group and therefore that this test is useful with a view to use as a diagnostic tool. These results clearly show the advantage of the present invention compared with the prior art since the present invention shows reproducibility that was not possible until then.

1-12. (canceled)

13. Method for the non-invasive evaluation of the physiopathological state of a biological tissue situated close to a joint, comprising the emission of an ultrasound wave in said tissue, from an ultrasound source, the simultaneous acquisition of at least one parameter extracted from the ultrasound signal received after propagation of the ultrasound wave, and of at least one joint force and/or movement data item, and the processing of the information simultaneously acquired; said method not being an ultrasound imaging method.

14. Evaluation method according to claim 13, further comprising a first step of applying a predefined joint force and/or movement.

15. Evaluation method according to claim 13, wherein the joint is a joint of the human body comprising a main degree of freedom.

16. Evaluation method according to claim 13, wherein the joint is a joint of the human body comprising a main degree of freedom, said joint being an elbow joint, a knee joint, an ankle joint, a metacarpophalangeal joint, a metatarsophalangeal joint or an interphalangeal joint.

17. Evaluation method according to claim 13, wherein the biological tissue studied is a tendon, a muscle, a ligament, a nerve or the skin.

18. Evaluation method according to claim 13, wherein the biological tissue studied is an Achilles tendon, a quadricipital tendon or a brachial triceps tendon.

19. Evaluation method according to claim 13, wherein the processing of said data comprises the extraction of at least one ultrasound parameter chosen from the speed of propagation of the ultrasound wave, the attenuation of the ultrasound wave at the point of reception of the wave, the amplitude of the ultrasound wave at the point of reception of the wave, the frequency of the ultrasound wave at the point of reception of the wave, or the change in the frequency spectrum of the ultrasound wave; and the correlation between the chosen parameter and the joint force and/or movement.

20. Device for evaluating the physiopathological state of a biological tissue situated close to a joint, for implementing a method for the non-invasive evaluation of the physiopathological state of a biological tissue situated close to a joint, comprising the emission of an ultrasound wave in said tissue, from an ultrasound source, the simultaneous acquisition of at least one parameter extracted from the ultrasound signal received after propagation of the ultrasound wave, and of at least one joint force and/or movement data item, and the processing of the information simultaneously acquired; said method not being an ultrasound imaging method; said device comprising:
a means for measuring the force and/or the joint movement;
an ultrasound sensor comprising at least one sending transducer and at least one receiving transducer;
a system for the simultaneous acquisition of the ultrasound data received after propagation of an ultrasound wave emitted in said tissue and the force and/or joint-movement data; and
a system for processing said data.

21. Evaluation device according to claim 20, wherein said means for measuring the force and/or the joint movement is an ergometer, a force sensor, a movement sensor, an accelerometer or a dynamometer;

22. Evaluation device according to claim 20, further comprising a means for applying a joint force and/or movement.

23. Evaluation device according to claim 20, further comprising a means for applying a joint force and/or movement, wherein said means for applying a joint force and/or movement is an actuator, a hydraulic actuator or a motor.

24. Evaluation device according to claim 20, comprising, so as to be portable, integrated and non-invasive, at least one means for measuring force and/or a joint movement, at least one ultrasound sensor, at least one acquisition system, at least one processing system and at least one self-contained energy source; an ultrasound sensor being placed on the skin opposite the biological tissue and a means for measuring a force and/or a joint movement being situated at the joint being acted on.

25. Evaluation device according to claim 20, wherein the at least one sending transducer and the at least one receiving transducer are aligned along the functional axis of the tissue being studied.

26. Evaluation device according to claim 20, wherein the acquisition and processing system comprises an acquisition card, a microprocessor, a data storage space and at least one item of data processing software.

27. Evaluation device according to claim 20, further comprising a module for displaying the data after processing and/or a data transmission means.
